(19) United States
(12) Patent Application Publication
(10) Pub. No.: US 2026/0047746 A1
(43) Pub. Date: Feb. 19, 2026

(54) MEDICAL MEASUREMENT SYSTEMS AND METHODS RELATED THERETO

(71) Applicant: Boston Scientific Scimed, Inc., Maple Grove, MN (US)

(72) Inventors: Kian S. LIM, Shrewsbury, MA (US); Longquan CHEN, Lexington, MA (US); Robert J. RIKER, Sewickley, PA (US); Niraj Prasad RAUNIYAR, Plymouth, MN (US)

(73) Assignee: Boston Scientific Scimed, Inc., Maple Grove, MN (US)

(21) Appl. No.: 19/293,180

(22) Filed: Aug. 7, 2025

Related U.S. Application Data
(60) Provisional application No. 63/682,970, filed on Aug. 14, 2024.

(51) Int. Cl. A61B 1/00; A61B 1/05; A61B 1/06; A61B 1/07; A61B 17/00; A61B 18/00; A61B 18/26

(52) U.S. Cl. CPC A61B 1/00006; A61B 1/00009; A61B 1/05; A61B 1/063; A61B 1/0655; A61B 1/0676; A61B 1/07; A61B 18/26; A61B 2017/00057; A61B 2018/00982

(57) ABSTRACT

A medical system includes a scope with a distal portion and a processor. The distal portion includes a first laser fiber, a second laser fiber, an imager, and one or more illumination devices. The processor is configured to cause the first laser fiber to project a first beam and the second laser fiber to project a second beam, cause the imager to capture one or more images of the target comprising a first image group, cause the medical system to transition to a first configuration, cause the imager to capture one or more images of the target comprising a second image group, and combine the first and second image groups to create a composite image. The first configuration includes one or more illumination devices in a first illumination state, the imager in a first exposure state, and the first and second laser fibers in a first laser state.

### CROSS-REFERENCE TO RELATED APPLICATION(S)

This application claims the benefit of priority to U.S. Provisional Application No. 63/682,970, filed on Aug. 14, 2024, which is incorporated by reference herein in its entirety.

### TECHNICAL FIELD

Aspects of this disclosure generally relate to medical systems and procedures. Particular aspects relate to medical systems and methods for measuring object size, for example, using one or more illumination devices.

### BACKGROUND

The manner in which one or more objects (e.g., urinary or kidney stones) may be removed from patients may depend on the size of the object(s). For example, some smaller stones, or fragments of stones, may be of an adequate size to pass through a bodily lumen (e.g., the urinary tract) and out of the body. However, some larger stones, or residual fragments of stones, may require removal via a procedure (e.g., an ureteroscopic procedure), such as retrieval by a retrieval device (e.g., an expandable basket, grasper, etc.), or further fragmentation into smaller pieces (e.g., via lithotripsy). Any residual fragments greater than 5 mm may further require follow-up re-intervention to remove such fragments from a patient. Thus, accurate stone size estimation or measurement may be an important aspect of stone removal and lithotripsy. However, both the fish-eye lenses used for endoscopic evaluation of stone size and the lack of visual size references at the appropriate depth of view often make accurate assessment of stone size difficult.

The current disclosure may help remedy one or more of these issues or other issues in the art.

### SUMMARY

According to an example, a method may include inserting a distal portion of a scope of a medical system into a bodily orifice or lumen. The distal portion of the scope may include a first laser fiber, a second laser fiber, an imager, and one or more illumination devices. The method may further include positioning the distal portion of the scope adjacent to a target within the bodily orifice or the lumen, projecting a first beam via the first laser fiber of the scope and a second beam via the second laser fiber of the scope, and capturing, via the imager, one or more images of the target including a first image group, and transitioning the medical system to a first configuration. The first configuration may include the one or more illumination devices being in a first illumination state, the imager being in a first exposure state, and the first laser fiber and the second laser fiber being in a first laser state capturing, via the imager, one or more images of the target including a second image group. The method may further include combining the first image group and the second image group to create a composite image.

Any of the examples described herein may have any of these features in any combination. Transitioning the medical system to the first configuration may include dimming or deactivating the one or more illumination devices to be in the first illumination state. Transitioning the medical system to the first configuration may further include deactivating the first laser fiber and the second laser fiber to be in the first laser state. The method may further include transitioning the medical system to a second configuration. The second configuration may include (i) the one or more illumination devices being in a second illumination state. Transitioning the medical system to the second configuration may include brightening or activating the one or more illumination devices to be in the second illumination state. The first image group may be captured with the medical system in the first configuration. A second configuration may include (i) the one or more illumination devices being configured with the second illumination state and (ii) the first laser fiber and the second laser fiber being in the first laser state. The second image group may be captured with the medical system in the second configuration.

The first laser fiber may be configured to transmit a first collimated beam and the second laser fiber may be configured to transmit a second collimated beam. The first collimated beam emitted from a distal end of the first laser fiber and the second collimated beam emitted from a distal end of the second laser fiber may be parallel to each other and may maintain a distance between each other that may be equivalent to a distance between a distal end of the first laser fiber and a distal end of the second laser fiber. The method may further include measuring a dimension of the target based on (i) a known distance between the first collimated beam and the second collimated beam and (ii) the composite image. The composite image may have a different exposure than either the first image group or the second image group. The method may further include transitioning the medical system to a third configuration. The third configuration may include (i) the imager being in a second exposure state. The method may further include capturing, via the imager, one or more images of the target including a third image group. The combining may further include the third image group. The first configuration may include (i) the one or more illumination devices being in the first illumination state and (ii) the first laser fiber and the second laser fiber being in the first laser state. The first image group may be captured with the medical system in the first configuration. The first laser state may include the first laser fiber and the second laser fiber each being in active states.

A second configuration of the medical system may include (i) the one or more illumination devices being in the first illumination state and (ii) the imager being in the first exposure state. The second image group may be captured with the medical system in the second configuration. The third configuration may include (i) the one or more illumination devices being in the first illumination state and (ii) the imager being in the second exposure state, and (iii) the first laser fiber and the second laser fiber being in a second laser state different from the first laser state. The third image group may be captured with the medical system in the third configuration. The first laser fiber may be configured to transmit a first collimated beam and the second laser fiber may be configured to transmit a second collimated beam. The first collimated beam may be emitted from a distal end of the first laser fiber and the second collimated beam may be emitted from a distal end of the second laser fiber. The first collimated beam and the second collimated beam may be parallel to each other and may maintain a distance between each other that may be equivalent to a distance between a distal end of the first laser fiber and a distal end of the second laser fiber. The method may further include measuring a dimension of the target, based on (i) a known distance between the first collimated beam and the second collimated beam and (ii) the composite image.

In another example, a method may include inserting a distal portion of a scope into a bodily orifice or lumen, positioning the distal portion of the scope adjacent to a first target within the bodily orifice or lumen, saving a first configuration including three parameters, the three parameters including a first exposure parameter, a first illumination parameter, and a first laser parameter. The method may further include acquiring a first view of the first target in a field of view of an imager, determining that the first view with the first configuration does not meet a predetermined quality metric, adjusting one of the parameters of the first configuration, saving a second configuration including the adjusted parameter of the first configuration and two unadjusted parameters of the first configuration, and capturing a first image group including one or more images of the first target with the second configuration.

Any of the examples described herein may have any of these features in any combination. The method may further include determining a second target from a predefined list of targets, calculating a third configuration based on the second target and the second configuration, acquiring a first view of the second target in the field of view of the imager, determining that the first view of second target with the third configuration does not meet the predetermined quality metric. Adjusting one of the parameters of the third configuration may be based on the first view of the second target. The method may further include saving a fourth configuration including the adjusted parameter of the third configuration and the two unadjusted parameters of the third configuration. The first view of the second target with the fourth configuration may meet the predetermined quality metric. The method may further include capturing a second image group. The second image group may include one or more images of the second target with the fourth configuration. The method may further include determining that there are no additional targets from the predefined list of targets, restoring the saved first configuration, and generating a first measurement based on the first image group and a second measurement based on the second image group.

In yet another example, a medical system may include a scope. The scope may include a handle and a shaft defining a channel having a distal opening. The medical system may further include a processor, a light source, at least one laser source, a first laser fiber, and a second laser fiber. Each of the first laser fiber and the second laser fiber may be coupled to the at least one laser source and extends through the shaft. A distal end of the shaft may include an imager, the distal opening of the channel, a distal end of the first laser fiber, and a distal end of the second laser fiber. The first laser fiber may be configured to transmit a first collimated beam onto a target, and the second laser fiber may be configured to transmit a second collimated beam on the target. The processor may be configured to adjust (i) a laser parameter for the first laser fiber and the second laser fiber, and (ii) an illumination parameter for the light source. The imager may be configured to capture a first image with the at least one laser source in a first laser parameter and the light source in a first illumination parameter. The imager may be configured to capture a second image with the at least one laser source in a second laser parameter different from the first laser parameter and the light source in a second illumination parameter different from the first illumination parameter. The processor may be configured to combine the first image and the second image into a third image.

Any of the examples described herein may have any of these features in any combination. The third image may have a different exposure than both the first image and the second image.

In yet another example, a medical system may include a scope. The scope may include a distal portion configured for insertion into a bodily orifice or lumen. The distal portion may be configured to be positioned adjacent to a target within the bodily orifice or lumen. The distal portion may include a first laser fiber, a second laser fiber, an imager, and one or more illumination devices. The medical system may further include a processor. The processor may be configured to cause the first laser fiber to project a first beam and the second laser fiber to project a second beam, cause the imager to capture one or more images of the target comprising a first image group, and may cause the medical system to transition to a first configuration. The first configuration may include the one or more illumination devices being in a first illumination state, the imager being in a first exposure state, and the first laser fiber and the second laser fiber being in a first laser state. The processor may be further configured to cause the imager to capture one or more images of the target comprising a second image group, and combine the first image group and the second image group to create a composite image.

Any of the examples described herein may have any of these features in any combination. Transitioning the medical system to the first configuration may include dimming or deactivating the one or more illumination devices to be in the first illumination state. Transitioning the medical system to the first configuration may further include deactivating the first laser fiber and the second laser fiber to be in the first laser state. The processor may be further configured to cause the medical system to transition to a second configuration. The second configuration may include (i) the one or more illumination devices being in a second illumination state. Transitioning the medical system to the second configuration may include brightening or activating the one or more illumination devices to be in the second illumination state. The first image group may be captured with the medical system in the first configuration. A second configuration may include (i) the one or more illumination devices being configured with the second illumination state and (ii) the first laser fiber and the second laser fiber being in the first laser state. The second image group may be captured with the medical system in the second configuration. The first laser fiber may be configured to transmit a first collimated beam and the second laser fiber may be configured to transmit a second collimated beam. The first collimated beam emitted from a distal end of the first laser fiber and the second collimated beam emitted from a distal end of the second laser fiber may be parallel to each other, maintaining a distance between each other that may be equivalent to a distance between a distal end of the first laser fiber and a distal end of the second laser fiber. The processor may be further configured to measure a dimension of the target based on (i) a known distance between the first collimated beam and the second collimated beam and (ii) the composite image. The composite image may have a different exposure than either the first image group or the second image group. The processor may be further configured to cause the medical system to transition to a third configuration. The third configuration may include (i) the imager being in a second exposure state. The processor may be further configured to cause the imager to capture one or more images of the target comprising a third image group. The combining may further include the third image group. The first configuration may include (i) the one or more illumination devices being in the first illumination state and (ii) the first laser fiber and the second laser fiber being in the first laser state. The first image group may be captured with the medical system in the first configuration. The first laser state may include the first laser fiber and the second laser fiber each being in active states. A second configuration of the medical system may include (i) the one or more illumination devices being in the first illumination state and (ii) the imager being in the first exposure state. The second image group may be captured with the medical system in the second configuration. The third configuration may include (i) the one or more illumination devices being in the first illumination state and (ii) the imager being in the second exposure state, and (iii) the first laser fiber and the second laser fiber being in a second laser state different from the first laser state. The third image group may be captured with the medical system in the third configuration. The first laser fiber may be configured to transmit a first collimated beam and the second laser fiber may be configured to transmit a second collimated beam. The first collimated beam emitted from a distal end of the first laser fiber and the second collimated beam emitted from a distal end of the second laser fiber may be parallel to each other, maintaining a distance between each other that may be equivalent to a distance between a distal end of the first laser fiber and a distal end of the second laser fiber. The processor may be further configured to measure a dimension of the target, based on (i) a known distance between the first collimated beam and the second collimated beam and (ii) the composite image.

It may be understood that both the foregoing summary and the following detailed descriptions are exemplary and explanatory only, neither being restrictive of the inventions claimed below.

### BRIEF DESCRIPTION OF THE DRAWINGS

The accompanying drawings are incorporated in and constitute a part of this application. These drawings illustrate aspects of the disclosure that, together with the written descriptions herein, serve to explain this disclosure. Each drawing depicts one or more exemplary aspects according to this disclosure, as follows:

FIG. 1A depicts an exemplary scope system, including an exemplary scope.

FIG. 1B depicts an exemplary distal end of the scope of FIG. 1A.

FIG. 1C depicts an exemplary distal face of the distal end shown in FIG. 1B.

FIGS. 2A-2C depict exemplary images obtained from the scope system of FIG. 1A.

FIG. 3 depicts a first exemplary flow diagram of an exemplary method.

FIGS. 4A-4C depict an exemplary process of augmenting an image from the scope of FIG. 1A.

FIG. 5 depicts a second exemplary flow diagram of an exemplary method.

FIG. 6 depicts an exemplary flow diagram illustrating a third exemplary method.

### DETAILED DESCRIPTION

In some techniques, where an imaging device is used to visualize a stone, a surgeon may manually estimate stone size by comparing an unknown dimension of the stone (e.g., a perceived maximum width) with a known dimension of a medical scope or accessory instrument (e.g., a diameter of an opening of a retrieval device). These estimations are often inaccurate, owing to the inherent challenges associated with measuring a three-dimensional object from a two-dimensional image, especially when the image is of low resolution or visibility, and endoscopic optics may cause image scaling to vary dramatically with relatively small changes in subject distance from camera. Because of these inaccuracies, the surgeon may be required to remove and/or further fragment a larger number of stones than medically required, increasing operation times. Even more time may be lost if the surgeon introduces a retrieval device based on the estimated stone size, then finds the stone or fragment too big for the device, requiring removal of the retrieval device and/or further fragmentation.

In addition to accurate stone size estimation, accurate measurement of the displacement or depth of the stone from a distal tip of the medical scope may also be important for related purposes. For example, for laser lithotripsy, the displacement of the stone from the lithotripsy device (which may extend from a distal end of the medical scope) may be relevant when determining the degree of laser energy to be delivered to fragment the stone. However, estimation of this displacement based on the imaging provided by the scope may often be inaccurate and unreliable, which in turn may affect the delivery of laser for lithotripsy purposes. The disclosed methods and systems may produce more accurate measurements of stones than existing methods and devices.

Aspects of the disclosure are now described with reference to exemplary systems and methods for measuring stone size and displacement. Some aspects are described with reference to medical procedures where a scope is guided through a body until a distal end of the scope is located in a body cavity including one or more stone objects. For example, the scope may include an elongated sheath that is guided through a urethra, a bladder, a ureter, and at least a portion of a kidney until a distal end of the sheath is located in or adjacent to a calyx of the kidney, adjacent one or more kidney stones. References to a particular type of procedure (e.g., a medical procedure); body cavity (e.g., a calyx); and an object (e.g., a kidney stone or a kidney stone fragment) are provided for convenience and not intended to limit the disclosure unless claimed. Accordingly, the concepts described herein may be utilized for any analogous device or method-medical or otherwise, kidney-specific or not.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Embodiments of the disclosure may help remedy one or more of the limitations in the art. The scope of the disclosure, however, is defined by the attached claims and not the ability to solve a specific problem.

The disclosure is drawn to medical systems including a medical device (e.g., a scope), including at least two laser transmitters or fibers at a distal end of the scope. This disclosure is also drawn to methods for performing medical procedures using the medical device. Each of the at least two laser transmitters is configured to deliver a parallel beam of light (e.g., a collimated beam), which may be projected or shone onto the targeted stone. As discussed in further detail below, such projection of the at least two collimated beams, via the scope, may help to accurately measure the size of the stone, as well as the displacement and/or distance of the stone from the distal end of the scope.

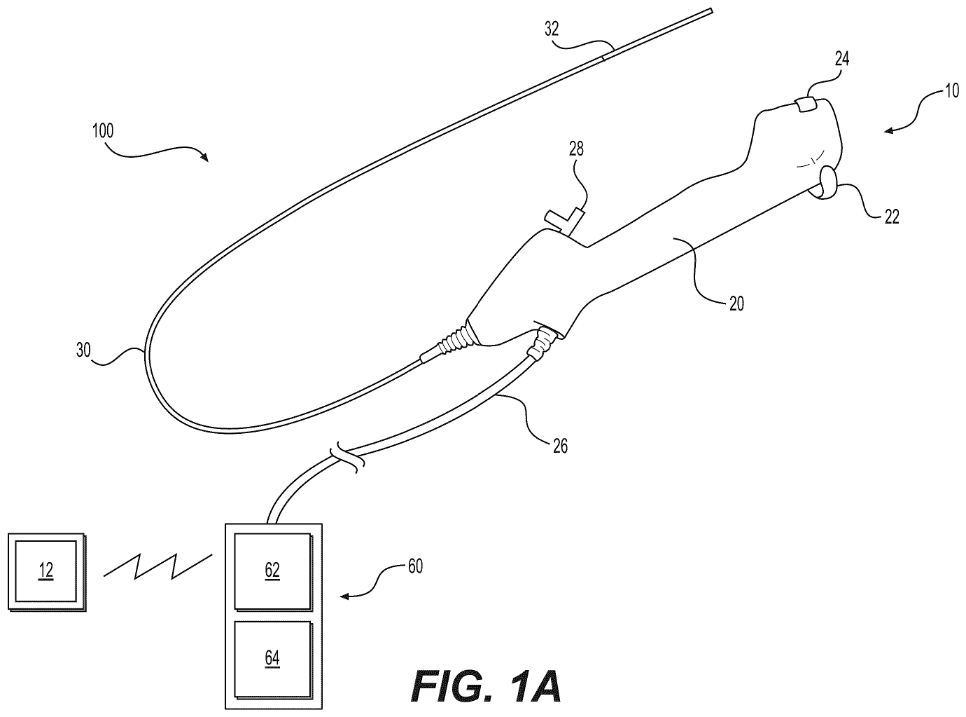

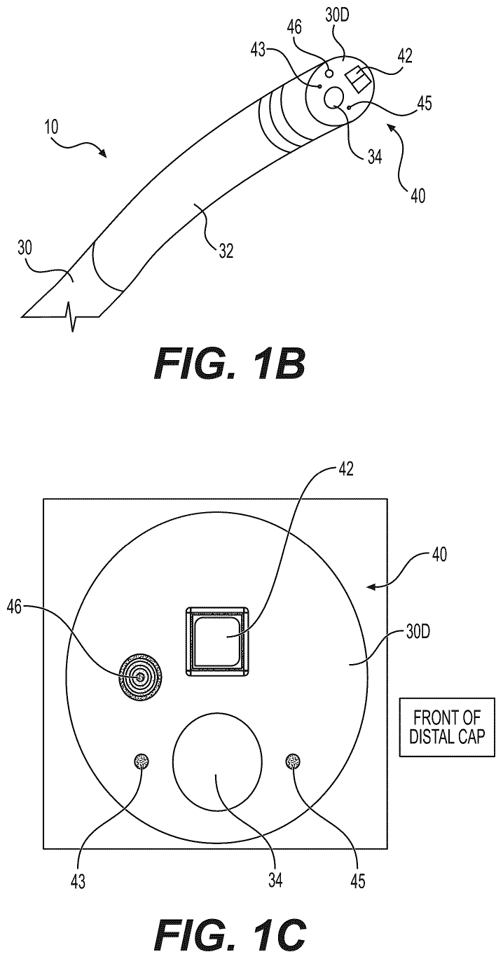

An exemplary system 100 is now described with reference to FIG. 1A. System 100 comprises a medical device, for example, a scope 10. Scope 10 may be in wired or wireless connection with one or more equipment supporting medical devices, for example, capital equipment or a tower 60. Scope 10 includes a handle 20 and an insertion portion or shaft 30. As discussed in detail below, a distal end 30D of scope 10 may include one or more lasers, imagers, light sources, and working channel distal openings. As shown, handle 20 may be coupled to tower 60 via at least one umbilicus. Alternatively, one or more components of tower 60 may be a part of scope 10, for example, stored within an internal portion of handle 20.

Tower 60 is not particularly limited. Tower 60 may be any suitable equipment, such as capital equipment, configured to be operable with scope 10, and to supply scope 10 with any necessary sources, for example, light, vacuum/suction, fluid, air/insufflation, power, signals, etc. As shown in FIG. 1A, tower 60 further includes at least a processing unit 62, which may be operable with an imaging unit of scope 10. Processing unit 62 may help generate a visual representation of the image data, and transmit the visual representation to one or more interface devices, for example, a screen 12. Processing unit 62 also may augment the visual representation. Screen 12 is not particularly limited and may be any suitable display, e.g., a touch-screen display, for displaying the image generated by the scope camera and processing unit 62.

Tower 60 may also include at least one laser 64. Laser 64 is not particularly limited, and may be any suitable laser device, e.g., one or more laser pointer(s), gas laser source(s), liquid laser source(s), semiconductor laser source(s), excimer laser source(s), laser diode(s), etc., as well as any suitable non-laser device, e.g., a LED with a collimator. In some examples, laser 64 may be of a small enough size so that laser 64 may be incorporated into scope 10 itself, as opposed to being in tower 60. In some examples, one laser 64 may generate a plurality (i.e., two or more) beams. In other examples, each laser 64 may generate one beam, and tower 60 may include multiple lasers 64. The intensity, hue, and other aspects of the beam generated by laser(s) 64 is not particularly limited, so long as (in at least one or more configurations or settings) the beam may be safely shone on a designated target stone (or other object or orifice), without altering (e.g., fragmenting) the target stone, as described below. In some examples, one or more lasers 64 may generate a first beam having a first color and a second beam having a second color. Laser(s) 64 may generate any suitable number of beams. All or some of the beams may have similar properties, or each of the beams may have unique properties. Laser(s) 64 may generate collimated beams, or other types of beams. Further properties of the beams from laser(s) 64 are discussed in further detail below.

Tower 60 may further include any additional equipment and/or devices that may be suitable or desired, including, for example, monitor(s), tubing(s), cable(s), etc. (not shown). Furthermore, the manner in which tower 60 and its various sources, equipment, etc. are arranged is not particularly limited. For example, though “tower” may imply a vertical arrangement of components/equipment used for support of scope 10, the arrangement of components/equipment may be in any suitable fashion, and the term “tower” is not limited to a particular arrangement.

Scope 10 of FIGS. 1A-1C is not particularly limited, and may be, as an example, any scope (e.g., bronchoscope, duodenoscope, endoscope, colonoscope, ureteroscope, etc.), catheter, tool, instrument, or the like, having a shaft/catheter that extends distally from a handle. In this example, scope 10 comprises: a handle 20 with a first actuator 22 and a second actuator 24; a port 28; and an elongated shaft 30 with a steerable portion 32.

Handle 20 is also not particularly limited, and may be any suitable medical device handle. First actuator 22 is not particularly limited, and may be any suitable control that may be operable, for example, to articulate steerable portion 32 by application of forces to the pull-wires contained within shaft 30. For example, first actuator 22 may include a lever, as shown in FIG. 1A. In alternatives, first actuator 22 may include, for example, a knob, a slider, a joystick, a button, or any other type of mechanism. Likewise, second actuator 24 is also not particularly limited, and may be operable for other suitable purposes, such as actuating or controlling other aspects of scope 10, e.g., turning on/off laser 64, performing image capture, or operating any tools or accessory instruments associated with scope 10. For example, second actuator 24 may include a button, as shown in FIG. 1A. In alternatives, second actuator 24 may include, for example, a knob, a slider, a joystick, a lever, or any other type of mechanism.

Cable 26 (e.g., an umbilicus), as depicted in FIG. 1A, is in connection with an aspect of tower 60. For example, a proximal end of cable 26 may terminate in a connector that is configured to mate with a socket of tower 60. Cable 26 is not particularly limited, and may include any suitable wiring/sheath suitable for various purposes. For example, cable 26 may be configured to sheath or otherwise enclose various cables and wirings (not shown) that transmit imaging data to processing unit 62 from imager 42 (shown in FIGS. 1B-1C). In addition (or alternatively), cable 26 may sheath or otherwise enclose laser fibers, such as a first laser fiber 43 and a second laser fiber 45. Laser fibers 43, 45 may include Holmium fibers, or other types of laser fibers or optical fibers with collimator. Each of first laser fiber 43 and second laser fiber 45 may be removably couplable to laser source(s) 64 via, for example, a connector at the proximal end of cable 26. For example, one or more of the connector of cable 26 or a socket of tower 60 may include a laser-to-fiber coupler. Such a coupler is not particularly limited, and may include any soldering, intermediary lenses, etc., which may assist with the coupling between fibers 43, 45 and laser source 64.

First laser fiber 43 and second laser fiber 45 (shown in FIGS. 1B and 1C), may each be configured to transmit, project, or otherwise emit a collimated beam (or other type of beam) from laser source 64 to distal ends of first laser fiber 43 and second laser fiber 45, respectively. As discussed in further detail below, first laser fiber 43 and second laser fiber 45 may extend through handle 20, towards a distal end 30D of shaft 30. Although two laser fibers 43, 45 are depicted in FIGS. 1B and 1C, it will be appreciated that any suitable number of laser fibers may be utilized. For example, three, four, five, or more laser fibers may be utilized. In at least some examples, it may be advantageous to include two or more laser fibers 43, 45.

As shown in FIG. 1A, port 28 may be mounted on a distal portion of handle 20, and include openings in communication with a working channel 34 (shown in FIGS. 1B-1C) of elongated shaft 30, discussed below. Any suitable accessory instrument/device or elongated tool, e.g., lithotripsy device, grasper, retrieval device, etc., may be inserted through port 28, and moved distally through the distal portion of handle 20 and/or working channel 34.

As shown in FIGS. 1A-1B, elongated shaft 30 extends distally from handle 20 before terminating at distal end 30D of shaft 30. Shaft 30 is not particularly limited, and may be any suitable flexible shaft configured to traverse bodily lumens during a procedure. As noted above, shaft 30 may include a steerable/articulating portion 32 that is adjacent to distal end 30D. Steerable/articulating portion 32 is not particularly limited, and may be any standard articulating portion of medical scope shafts.

Shaft 30 may further include at least one working channel 34, which may extend longitudinally from port 28 of handle 20 to distal end 30D of shaft 30. Shaft 30 may also include at least one lumen(s) for receiving any number of additional wirings, cables, and/or fibers from tower 60 that support the various features present on distal end 30D of shaft 30, which are discussed in further detail below. Laser fibers 43, 45 may extend from cable 26, through an interior of handle 20, and through shaft 30. For example, laser fibers 43, 45 may extend through lumen(s) of shaft 30 or through a central opening defined by an outer sheath of shaft 30. For example, each of first laser fiber 43 and second laser fiber 45 may include a single, continuous, monolithic piece of fiber extending from the connector of cable 26 to distal end 30D. In alternatives, one or both of first laser fiber 43 and second laser fiber 45 may include multiple pieces or segments that are in optical communication with one another (e.g., joined by connectors or fused together).

As shown in FIGS. 1B-1C, distal end 30D of shaft 30 may include at least a distal opening of working channel 34, imager 42, a light 46, and distal ends of laser fibers 43, 45. In other examples, shaft 30 may further include a distal cap (not shown) covering portions of distal end 30D, thereby protecting features such as imager 42, light 46, and laser fibers 43, 45.

The distal opening of working channel 34 is not particularly limited, and may be of any suitable width to enable the extension/retraction of an instrument or tool within/out of working channel 34. Imager 42 is also not particularly limited, and may be any suitable device configured to receive imaging data (e.g., via any sensor) of a designated target (e.g., a stone), and transmit the imaging data to an image processor (e.g., processing unit 62) for producing a visual representation of the imaging data. In some examples, imager 42 may be a camera including a CMOS sensor. In other examples, imager 42 may include fiber optics in communication with a sensor or other device that may be positioned within shaft 30 or handle 20. However, imager 42 is not limited to the aforementioned examples. Light 46 is also not particularly limited and may be any suitable device configured to provide a source of light, particularly when shaft 30 is traversing bodily lumens and imager 42 is capturing imaging data of a designated target. In some examples, light 46 may include a plastic optical fiber (“POF”) device configured to provide light from a light source of tower 60 (e.g., an LED, or an LED mounted at distal end 30D).

As shown in FIGS. 1B-1C, the distal tips/ends of first laser fiber 43 and second laser fiber 45 may be present on distal end 30D of shaft 30. As discussed above, while two laser fibers are depicted in shaft 30, the number of laser fibers is not particularly limited, so long as there is available space within shaft 30 and/or on distal end 30D to accommodate the additional laser fibers. In some examples, although not shown, system 100 may further include a third laser fiber, a fourth laser fiber, etc. As discussed above, laser fibers 43 and 45 are not particularly limited, and may be any suitable fiber for laser transmission. As discussed above, fibers 43 and 45 may be in communication with laser source 64 of tower 60. Laser source(s) 64 may transmit, emit, or project a collimated beam, which may travel through fibers 43, 45. Fibers 43 and 45 may be on opposing sides (e.g., diametrically opposite sides) of the distal opening of working channel 34. For example, fibers 43 and 45 may each be adjacent to the distal opening of working channel 34, on opposite sides of the distal opening of working channel 34. A line drawn through fibers 43 and 45 may approximately intersect a central axis of the distal opening of working channel 34. However, the position of fibers 43 and 45 on distal end 30D is not limited thereto.

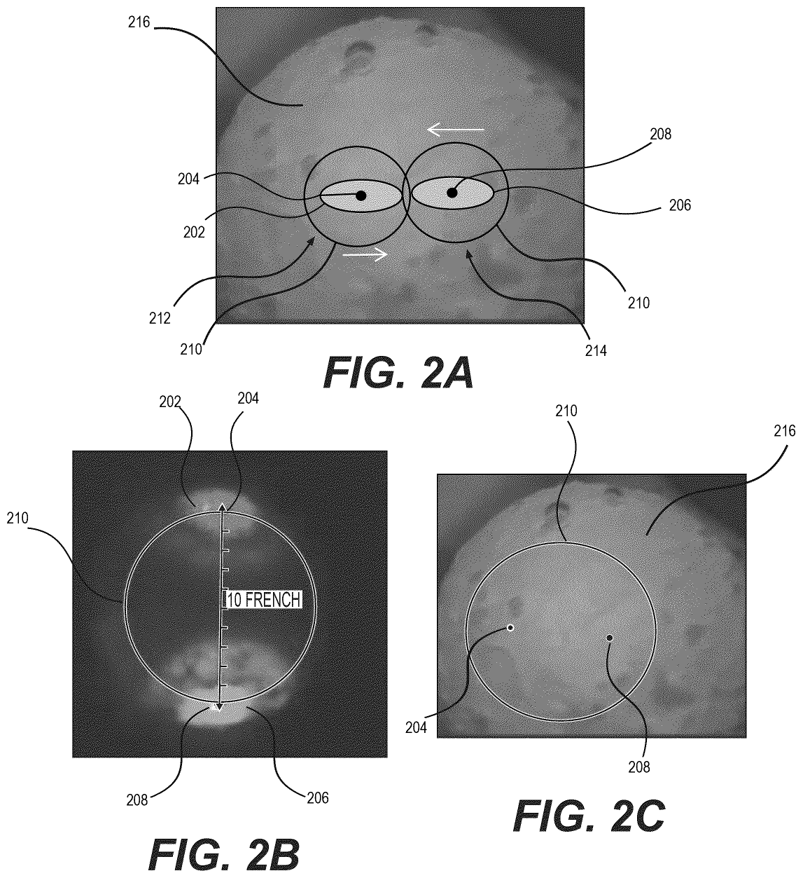

FIGS. 2A-2B depict exemplary images obtained from scope 10 of FIG. 1A. FIG. 2A depicts an example image showing white-washed (e.g., overexposed) laser dots. FIG. 2A shows a lighting area 202 and a lighting area 206. Lighting area 202 may include a center point 204, and lighting area 206 may include a center point 208. Each lighting area 202, 206 may correspond with emitted laser light from laser fibers 43 and 45. For example, light emitted by laser fiber 43 may correspond with lighting area 202, and light emitted by laser fiber 45 may correspond with lighting area 206. As previously noted, any number of laser fibers may correspond with any number of lighting areas (e.g., a third laser fiber may correspond with a third lighting area). Lighting areas 202 and 206 are depicted as approximately elliptical, but this is only exemplary and other shapes are possible.

It should be understood that each lighting area may correspond with different characteristics of light emitted by each of laser fibers 43 and 45. For example, laser fiber 43 may emit light at a first wavelength (e.g., color) and intensity, and laser fiber 45 may emit light at a second wavelength and intensity. This may result in a different visual appearance to a user of lighting area 202 and lighting area 206. For example, lighting area 202 may be green (corresponding to green light emitted by laser fiber 43), and lighting area 206 may be red (corresponding to red light emitted by laser fiber 45). Laser fibers 43 and 45 and lighting areas 202 and 206 are not particularly limited to any particular wavelength or intensity, and may be any standard wavelength or intensity.

During use of laser fibers and aiming beams for stone (or object or orifice) sizing procedures, the intensity of an aiming beam may create one or more artifacts in the glass of the one or more laser fibers. Additionally, the aiming beams themselves (e.g., light emitted by laser fibers 43 and 45) may oversaturate imager 42, resulting in an undesirable white dot(s) (e.g., blown-out highlights) in resulting images. Another potential source of overexposure is light emitted from light 46. Light emitted from light 46 (e.g., ambient light) may illuminate the scene (e.g., the target site) with approximately white light. The illuminated portion of the target site that is lit by light 46 may be referred to as illuminated area 216. Illuminated area 216 may interfere with lighting areas 202 and 206 due to all three light sources being aimed at approximately the same area at the target site. The appearance of blown-out highlights in the image on uneven and/or tortuous patient anatomy may make it difficult to calculate the position of center points 204 and 208, or to differentiate the center points 204 and 208 from other imaging artifacts.

Without accurate positions of center points 204 and 208, extrapolation of the edges of laser fibers 43 and 45 to their corresponding laser dots (corresponding approximately to lighting areas 202 and 206) may become inaccurate. For example, positioning of a metrology circle 210 may become difficult or impossible. FIG. 2A shows metrology circle 210 at a first position 212, approximately centered on center point 204. FIG. 2A also shows metrology circle 210 at a second position 214, approximately centered on center 208. Metrology circle 210 may drift, fluctuate, jump, or otherwise move between first position 212 and second position 214 (shown in FIG. 2A by the two white arrows) due to system 100 being unable to correctly position metrology circle 210 due to the aforementioned difficult lighting conditions. As will be discussed below, positioning metrology circle 210 at first position 212 and/or second position 214 may result in incorrect measurements. It follows that system 100 should calculate a third (e.g., correct) position for metrology circle 210.

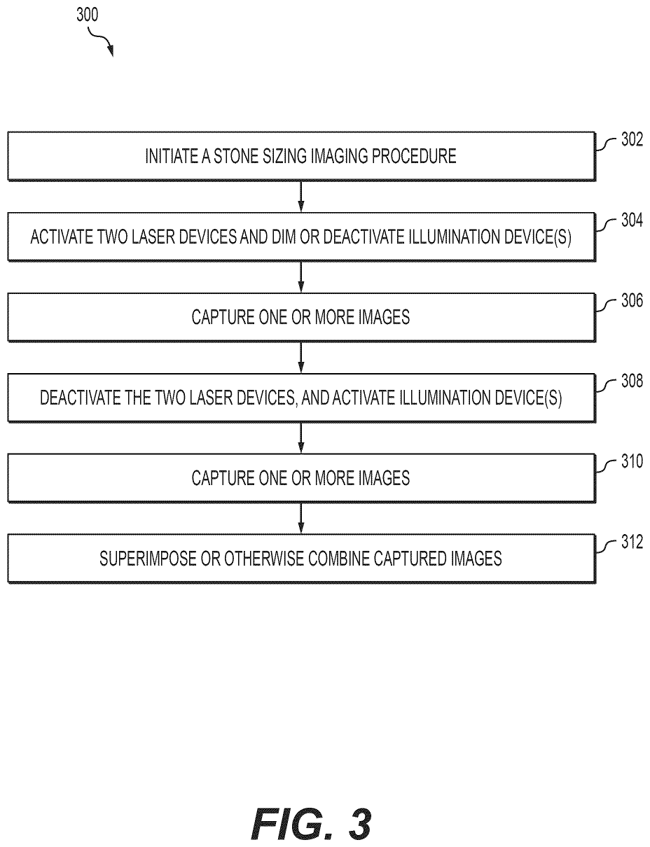

In one aspect of the disclosure, a first method is disclosed herein. For example, FIG. 3 illustrates an exemplary method 300 for optimized imaging exposure during a medical procedure. In a step 302, a user may initiate a stone sizing procedure with system 100. Initiation of the stone sizing procedure may correspond with the user actuating one or more actuators (e.g., first actuator 22), which may initiate a stone sizing procedure with processing unit 62. This example is only exemplary and other mechanisms of initiating the stone sizing procedure are within the scope of the disclosure. It should be understood that any method described herein may include inserting a scope (e.g., scope 10) into a bodily orifice or lumen and positioning scope 10 adjacent to a target within the bodily orifice or lumen.

In a step 304, system 100 may activate two laser devices (e.g., laser fibers 43 and 45), and dim or deactivate light levels of illumination device(s) (e.g., light 46). FIG. 2B shows an image depicting steps 304 and 306. Of note in FIG. 2B, and in contrast to FIG. 2A, is the absence of illuminated area 216 due to light 46 being turned off. It will be understood that light 46 does not always need to be turned completely off, and in some instances, may instead be dimmed.

FIG. 2B illustrates lighting areas 202 and 206, which are emitted by laser fibers 43 and 45, respectively. In the absence of illuminated area 216, lighting areas 202 and 206 are more readily visible to imager 42. In the example of FIG. 2B, center points 204 and 206 are separated by a distance of approximately 10 French (≈3.333 mm), though this is only exemplary and other distances are possible. The approximately 10 French distance between center points 204 and 206 may also define a diameter of metrology circle 210. Unlike the example shown in FIG. 2A, metrology circle 210 may not drift, fluctuate, jump, or otherwise move in the example of FIG. 2B because light 46 has been turned off, eliminating illuminated area 216. It should be understood that while metrology circle 210 is shown in FIG. 2B and described with respect to step 304, this is only exemplary. As will be described below, metrology circle 210 may be omitted from step 304 and may be introduced at a later step without departing from the scope of this disclosure.

In a step 306, system 100 and imager 42 may capture one or more images. As previously noted, the ambient lighting provided by light 46 (e.g., illuminated area 216) is not present at step 306, which may allow imager 42 to capture a clear image of lighting areas 202 and 206 without the interference of illuminated area 216. The one or more images captured at step 306, and any other image capture operation throughout this disclosure, may be described as an imaging group (e.g., image capture is not limited to a single image).

In a step 308, system 100 may deactivate the two laser devices (e.g., laser fibers 43 and 45), and may then activate the illumination device(s) (e.g., light 46). The ambient lighting provided by light 46 may illuminate area 216 once again.

In a step 310, system 100 may capture one or more images. The images captured in step 310 lack lighting areas 202 and 206, but do contain illuminated area 216. The images taken during step 310 may generally show the target site under normal (e.g., ambient) lighting conditions that a user may typically observe while navigating distal end 30D through patient anatomy and at the target site.

In a step 312, system 100 may superimpose or otherwise combine captured images (e.g., from steps 306 and 310). System 100 may generate metrology circle 210 based on the images taken in step 306, and superimpose metrology circle 210 onto the images taken in step 310 (e.g., the images illuminated by light 46). Such a composite image is shown in the example of FIG. 2C. FIG. 2C includes superimposed center points 204 and 208 in the final resulting image, though this is only exemplary. Metrology circle 210 is shown in FIG. 2C correctly positioned around center points 204 and 208 (in contrast to positions 212 and 214 shown in FIG. 2A). In some aspects, lighting areas 202 and 206 may be emphasized (e.g., brightened or decreased in transparency) in the composite image. In some aspects, lighting areas 202 and 206 may be deemphasized or otherwise removed from the resulting composite image. The composite image may have different brightness, or exposure, or one or more other image characteristics as compared to either the first image group or the second image group.

Steps 304-310 are preferably performed in rapid succession (e.g., approximately 0.1 seconds or less between each image capture), such that the resulting images from steps 306 and 310 are aligned (e.g., the target site, system 100, operator, and/or patient do not move between capture of the images). It should be understood that while FIG. 3 shows step 304 (e.g., lasers on, light(s) off) occurring before step 308 (e.g., lasers off, light(s) on), this is only exemplary. In some aspects, steps 308 and 304 may be switched in the order of steps without departing from the scope of this disclosure. In some aspects of the disclosure, various steps may be repeated and/or iterated.

In another aspect of the disclosure, systems, devices, and methods of optimization of laser fiber edge segmentation and extrapolation are disclosed herein. When capturing an image during a medical procedure with imager 42, at least three parameters may be adjusted to change the resulting image. These parameters may include: ambient light level (e.g., provided by light 46), exposure time, and laser beam intensity. Various combinations of the aforementioned three parameters may be experimentally determined to be optimal for a desired view, for example, of the laser fiber edge, laser aiming dot, and/or features at the target site. For example, a first combination of settings for the three parameters may be referred to as combination A, a second (different and/or unique) combination of the three parameters may be referred to as combination B, and a third (different and/or unique) combination of the three parameters may be referred to as combination C. The aforementioned combinations A-C may be preprogrammed to system 100, or may be adjusted by a user. For example, a user may experimentally determine that a given combination of the three parameters is preferable than or otherwise optimized compared to an existing combination of parameters for a given use case, and may reassign the parameters of an existing combination or create a new combination (e.g., combination D) with the newly selected parameters.

Throughout this disclosure, any reference to adjusting a parameter may also be referred to as adjusting a state. System 100 may also be referred to as being in a particular configuration. A configuration may include one or more imaging components being in respective particular states. For example, a first configuration may include states 1, 2, and 3, and a second configuration may include states 4, 5, and 6. It should be understood that configurations may differ from one another by at least one state. For example, a first configuration may include states 1, 2, and 3, and a second configuration may include states 1, 2, and 4. System 100 (and any subcomponent thereof) may be referred to as transitioning to/from a configuration to another configuration (e.g., by changing at least one parameter or state).

When a user initiates a stone sizing imaging procedure, multiple image frames may be captured with the programmed combinations of the three parameters (e.g., images may be taken with combinations A-C). After the imaging sequence has been completed, ambient light level, exposure time, and laser beam intensity may be returned to their clinical settings. System 100 may then perform various calculations for laser and fiber positions based on the multiple exposure conditions captured with the three different combinations, as well as the differences between them, thereby improving imaging accuracy. A composite image may have different brightness, or exposure, or one or more other image characteristics as compared to images taken with different combinations of parameters.

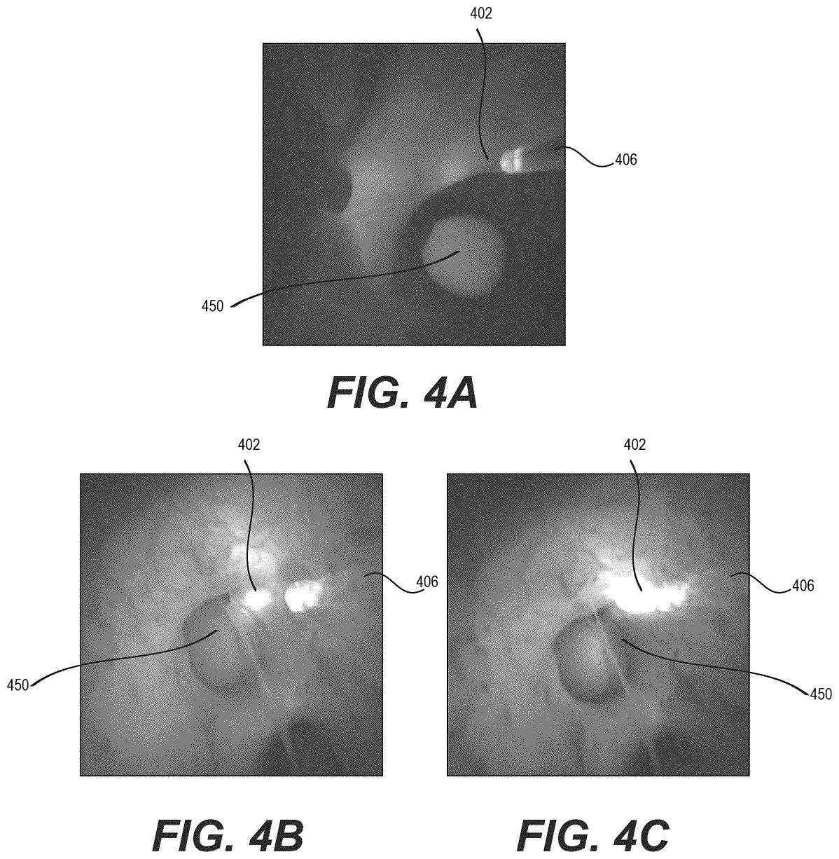

As an example, FIGS. 4A-4C may each show the same target site with different combinations (A-C) of the aforementioned parameters. Further shown in FIGS. 4A-4C is a stone 450, which may be imaged and sized by system 100 in preparation for a stone removal procedure. It is noted that although various aspects of this disclosure discuss imaging or sizing of a stone (e.g., stone 450), various aspects of this disclosure may be employed for imaging or sizing of another object, orifice, etc. It will be apparent that certain target and/or target site and/or system 100 features may be more or less visible in each of the FIGS. 4A-4C. As an example, a lighting area 402 (corresponding to a laser fiber 406) and laser fiber 406 may each have a greater visibility to imager 42 under one combination of parameters (e.g., shown in FIG. 4A) than compared to other combinations of parameters (e.g., shown in FIGS. 4B-4C). Lighting area 402 may have any of the characteristics of either of lighting areas 202 or 206. It should be noted that in some aspects, combinations of parameters may differ with respect to only a single parameter. For example, ambient light level and exposure time may be the same between two combinations, but laser beam intensity may differ.

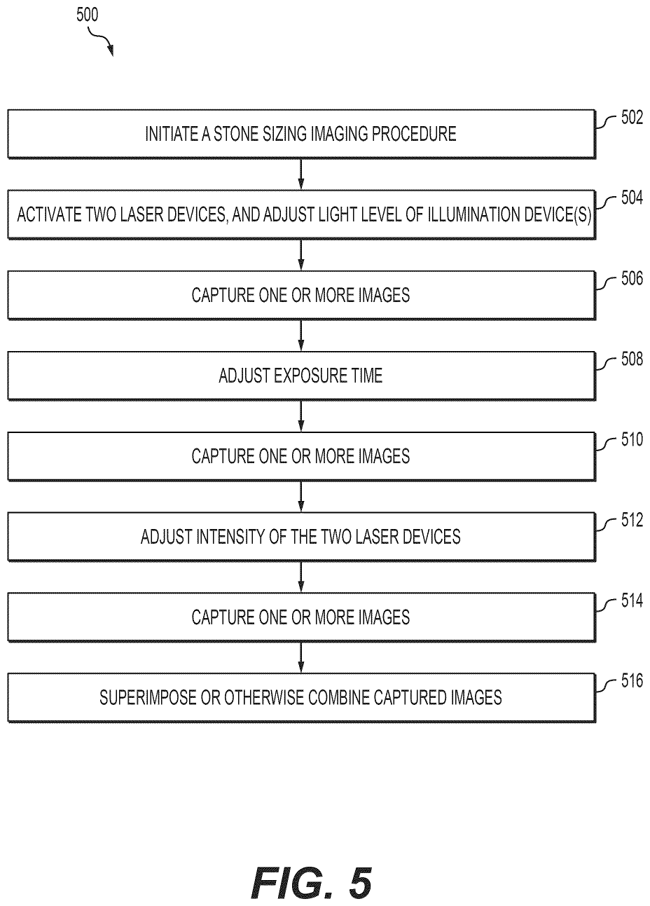

A method corresponding to another aspect of the disclosure is disclosed herein and shown in FIG. 5. For example, FIG. 5 illustrates a method 500 for optimized medical image capture, according to aspects of the disclosure.

In a step 502, a user may initiate a stone sizing procedure with system 100. Initiation of the stone sizing procedure may correspond with the user actuating one or more actuators (e.g., first actuator 22), which may initiate a stone sizing procedure with processing unit 62. It should be understood that this is only exemplary, however, and other mechanisms of initiating the stone sizing procedure are within the scope of the disclosure.

In a step 504, system 100 may activate two lasers (e.g., laser fibers 43 and 45), and adjust the light level (e.g., luminosity) of one or more illumination devices, such as light 46. The adjusted ambient light level, exposure time, and laser beam intensity in step 504 may be referred to a combination A.

Method 500 may include capturing one or more images with aspects of system 10 under various different configurations, parameters, states, etc. In a step 506, system 100 (via imager 42) captures one or more images using the settings of combination A (e.g., adjusted ambient light level, exposure time, and laser beam intensity). In a step 508, system 100 adjusts the exposure time parameter. The new combination of adjusted ambient light level, adjusted exposure time, and laser beam intensity may be referred to as combination B. In a step 510, system 100 (via imager 42) captures one or more images using the settings of combination B (e.g., adjusted ambient light level, adjusted exposure time, and laser beam intensity).

In a step 512, system 100 adjusts the intensity of the two laser devices, laser fibers 43 and 45. The new combination of adjusted ambient light level, adjusted exposure time, and adjusted laser beam intensity may be referred to as combination C. In a step 514, system 100 (via imager 42) captures one or more images using the settings of combination C (e.g., adjusted ambient light level, adjusted exposure time, and adjusted laser beam intensity).

In a step 516, system 100 superimposes or otherwise combines the captured images taken with combinations A, B, and C. The superimposed or otherwise combined image may have optimized visibility of various parts of the imaging scene as compared to each of the individual image(s) taken with combinations A, B, and C.

In yet another aspect of the disclosure, systems, devices, and methods of dynamic optimization of laser fiber edge segmentation and extrapolation are disclosed herein. Ambient light levels, exposure time, and laser beam intensity may be rapidly and dynamically adjusted to optimize the detectability of laser fiber position(s), the aim laser, and/or uroliths at the target site. In this embodiment, system 100 may respond to dynamic clinical conditions by performing real-time optimization of the settings (e.g., parameters) separately with respect to each of the detection targets.

When a user initiates the stone sizing algorithm, system 100 may image a sequence of imaging targets (e.g., aim laser, fiber jacket(s), fiber edges, fiber tip(s), and uroliths). For each of these targets, system 100 may compare the imaging frame (e.g., including the target) to a quality metric (e.g., whether the imaging frame including the targets meets the quality threshold). The quality metric may help to define or quantify how well each target is defined in a given image. Any of a variety of rapid optimization algorithms (e.g., simplex) may be employed to adjust the exposure parameters for the best target detection quality. In some aspects, the quality metric may be predetermined.

As system 100 proceeds to the next imaging target, the exposure(s) from previous target(s) may be implemented to help speed up optimizations. Subsequent target quality measurements may also be informed by the identification of previous target locations. The optimizations may preferably be completed in a short period of time in response to the user-requested stone sizing, so as not to interfere with normal clinical imaging. Similarly to the embodiment of the disclosure discussed above with respect to FIG. 5, further enhancements to detectability of some targets may be derived from image analysis of multiple frames (e.g., more accurate center point(s) of the aim laser(s) may be calculated).

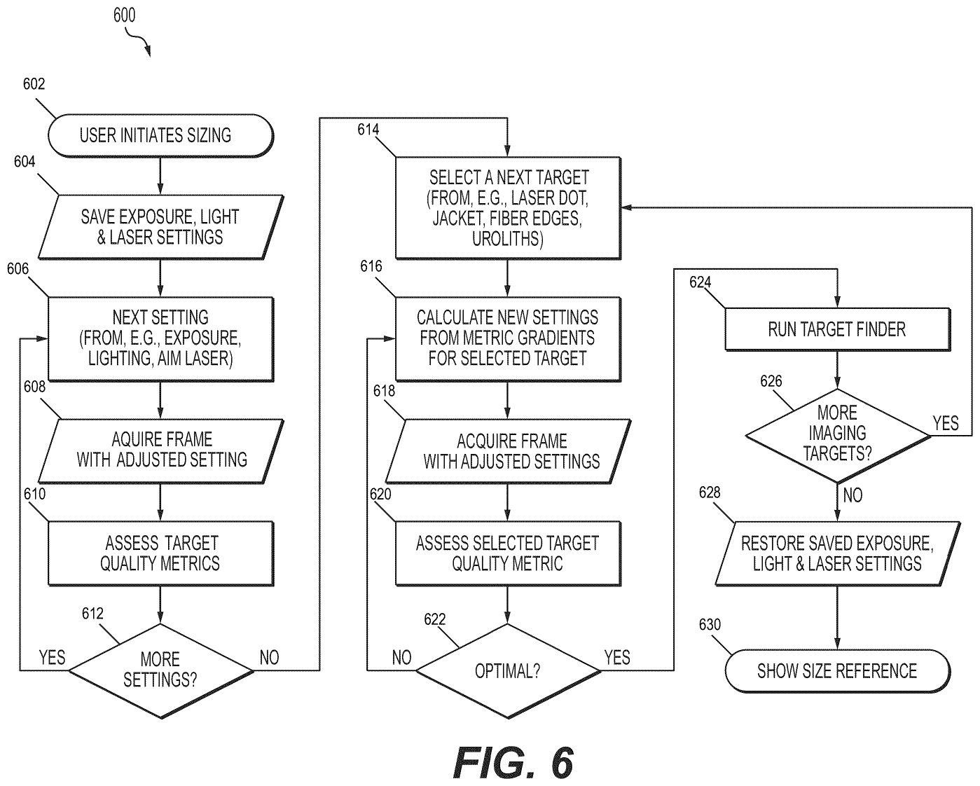

A method 600 corresponding to the third aspect of the disclosure is disclosed herein and shown in FIG. 6. For example, FIG. 6 illustrates method 600 for dynamically adjusting medical imaging parameters, according to aspects of the disclosure.

In a step 602, a user initiates a stone sizing procedure with system 100. Initiation of the stone sizing procedure may correspond with the user actuating one or more actuators (e.g., second actuator 24), which may initiate a stone sizing procedure with processing unit 62. It will be understood that this is only exemplary, however, and other mechanisms of initiating the stone sizing procedure are within the scope of the disclosure.

In a step 604, system 100 saves current parameters for ambient light level, exposure time, and laser beam intensity. The saved parameters may be referred to as a first setting.

In a step 606, system 100 adjusts one of the aforementioned parameters of the first setting for ambient light level, exposure time, and laser beam intensity. For example, system 100 may adjust the ambient light level, without changing exposure time or laser beam intensity. The updated parameters may be referred to as a second setting.

In a step 608, system 100 acquires a viewing frame with the adjusted second setting.

In a step 610, system 100 assesses target quality metrics. For example, system 100 may assess one or more quality metrics for the current imaging target. Quality metrics may vary based on imaging target. For example, a laser dot metric may include a number of contiguous pixels within a specified hue range and greater than a specified brightness threshold. Quality metrics may include information related to location of other imaging targets. For example, a position of a laser dot may influence system 100's assessment of the quality of an image with respect a laser fiber edge.

In a step 612, system 100 determines whether all of the adjustable settings have been manipulated. For example, in step 606, system 100 cycles through the list of exposure, lighting, and aim laser. If not all of the settings of step 606 have been adjusted (e.g., exposure and lighting have been adjusted, but aim laser has not), system 100 returns to step 606.

In some aspects, system 100 may determine whether the current setting (e.g., the second setting) produces an image of the target that satisfies the quality metric. In such a case, system 100 may determine that additional adjustment of one or more parameters is needed (e.g., “YES” in FIG. 6). System 100 may return to step 606 and may adjust the same parameter previously adjusted, or may adjust a different parameter. In some aspects, system 100 may adjust more than one setting at the same time during step 606, for example, when repeating step 606. The updated setting may be referred to as a third setting. It will be understood that states that are not adjusted in a given configuration may be referred to as unadjusted states. For example, if there are states 1, 2, and 3, and state 1 is adjusted to state 1*, states 2 and 3 may be referred to as unadjusted states.

It should be noted that while steps 606, 608, 610, and 612 are depicted as discrete steps in FIG. 6, in some aspects of the disclosure, these steps may be parts of a single continuous step. As part of a single step, system 100 may cycle through imaging frames (and assess the quality metrics of those imaging frames) by adjusting exposure, lighting, and aim laser settings individually (e.g., one after the other). Once all three of exposure, lighting, and aim laser settings have been adjusted, system 100 may then proceed to a step 614.

If system 100 determines that the current lighting setting settings do produce an image of the target that satisfies the quality metric, then system 100 may proceed to step 614. That is, if system 100 determines that the current lighting settings do not need additional adjustments of one or more parameters (e.g., “NO” in FIG. 6), then system 100 may proceed to step 614.

In step 614, system 100 (e.g., imager 42) may select a next imaging target, such as a laser dot, a laser fiber jacket, a laser fiber edge, a urolith, etc. In some aspects, the next imaging target may be selected from a predefined list by either the user or system 100.

Steps 616, 618, 620, and 622 may be performed by system 100 rapidly with no user input or guidance. Next, in a step 616, system 100 calculates new settings from metric gradients for the newly selected target. For example, system 100 may apply one or more previously determined settings (e.g., the settings used when the answer to step 612 is “NO”). It should be noted that settings calculated in step 616 may be calculated contemporaneously and may be independent of previously calculated settings.

In a step 618, system 100 acquires the imaging frame with the adjusted parameters.

In a step 620, system 100 may assess an overall quality metric for the newly acquired imaging target. In an example, system 100 may use one or more rapid optimization algorithms, such as simplex to assess the quality metric of the image of the newly acquired target.

In a step 622, system 100 may determine whether the overall target image quality metric from step 620 is optimal (e.g., are greater than or equal to a predetermined quality metric or whether new settings generated in step 616 will improve image quality below a predefined increment). If the assessed overall target image quality metric from step 622 is not optimal (e.g., “NO” in FIG. 6), system 100 returns to step 616 and calculates one or more new parameters from metric gradients for the current target, and saves the result as a fourth setting. If the assessed overall target image quality metric from step 622 is optimal (e.g., “YES” in FIG. 6), system 100 proceeds to a step 624. System 100 may use one or more rapid optimization algorithms to evaluate whether quality metrics are optimal, such as such as simplex or gradient descent algorithms.

In step 624, system 100 may initiate a target location operation. System 100 may identify one or more potential targets in the imaging frame and proceed to a next target based on a predefined list or user input. For example, system 100 may employ machine vision techniques to locate targets. For example, system 100 may recognize that a particular feature of the imaging frame is a laser fiber. In some aspects, system 100 may label or mark the identified feature on a user interface, such as a display, touch screen, etc.

In a step 626, system 100 may determine, based on the configured list of targets needed to calculate a size reference in the current clinical workflow, whether there are additional imaging targets that need to be located. If there are additional targets that need to be imaged (e.g., “YES” in FIG. 6), system 100 may return to step 614 and may select a next imaging target, such as a laser dot, a laser fiber jacket, a laser fiber edge, a urolith, etc. If there are not additional targets that need to be imaged (e.g., “NO” in FIG. 6), system 100 may proceed to step 628 and restore (e.g., revert back to) the saved ambient light level, exposure time, and laser beam intensity parameters from step 604 (e.g., the first setting).

In a step 630, system 100 may indicate or otherwise show a size reference (e.g., provide the calculated stone size to the user). The size reference may be displayed on a user interface for analysis by a user. For example, a user may determine that a given stone is too large to be passed naturally out of the patient, and instead must be removed or broken up (e.g., via a fragmentation procedure). The user interface may associate certain stone sizes with certain graphic user interface elements. For example, stone sizes that may be safely passed through the patient without outside intervention may be marked with a certain color (e.g., green) and indicated as safe, acceptable etc. Similar techniques may be applied to stones of moderate and large size. In another example, system 100 may provide a user with an ordered list on the user interface of measured features during a given imaging procedure. In such an example, system 100 may group all measured stones and list them by size (e.g., ascending, descending).

The systems, devices, and methods disclosed herein may provide optimized medical imaging measurements. A user may apply the systems, devices, and methods of this disclosure to more accurately and precisely measure features of the human body, such as a kidney stone. The systems, devices, and methods of this disclosure may reduce measurement errors caused by poor lighting conditions within various internal lumens, passages, organs, and orifices of the human body. Optimized measurement of internal features may result in better procedure planning and patient outcomes.

The systems, devices, and methods of the disclosure may help to rapidly modify medical imaging conditions to support accurate and precise measurements. The systems, devices, and methods of the disclosure may help to enhance clinical target finding. Target finding may be beneficial when evaluating one or more measurements by providing system 100 with additional known locations of various features (e.g., a laser dot, a laser fiber, etc.) to consolidate when calculating a stone sizing measurement. While reference in this disclosure is made to stone sizing measurements, the systems, devices, and methods of the disclosure may be applicable to various other anatomical features and/or medical devices and instruments present inside of a human body.

While principles of the disclosure are described herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A method comprising:
inserting a distal portion of a scope of a medical system into a bodily orifice or lumen, wherein the distal portion of the scope includes a first laser fiber, a second laser fiber, an imager, and one or more illumination devices;
positioning the distal portion of the scope adjacent to a target within the bodily orifice or the lumen;
projecting a first beam via the first laser fiber of the scope and a second beam via the second laser fiber of the scope;
capturing, via the imager, one or more images of the target comprising a first image group;
transitioning the medical system to a first configuration, wherein the first configuration comprises the one or more illumination devices being in a first illumination state, the imager being in a first exposure state, and the first laser fiber and the second laser fiber being in a first laser state;
capturing, via the imager, one or more images of the target comprising a second image group; and
combining the first image group and the second image group to create a composite image.

2. The method of claim 1, wherein transitioning the medical system to the first configuration includes dimming or deactivating the one or more illumination devices to be in the first illumination state.

3. The method of claim 2, wherein transitioning the medical system to the first configuration further includes deactivating the first laser fiber and the second laser fiber to be in the first laser state.

4. The method of claim 3, further comprising:
transitioning the medical system to a second configuration, wherein the second configuration comprises (i) the one or more illumination devices being in a second illumination state, wherein transitioning the medical system to the second configuration includes brightening or activating the one or more illumination devices to be in the second illumination state.

5. The method of claim 4, wherein the first image group is captured with the medical system in the first configuration.

6. The method of claim 5, wherein a second configuration comprises (i) the one or more illumination devices being configured with the second illumination state and (ii) the first laser fiber and the second laser fiber being in the first laser state, wherein the second image group is captured with the medical system in the second configuration.

7. The method of claim 6, wherein the first laser fiber is configured to transmit a first collimated beam and the second laser fiber is configured to transmit a second collimated beam, and wherein the first collimated beam emitted from a distal end of the first laser fiber and the second collimated beam emitted from a distal end of the second laser fiber are parallel to each other, maintaining a distance between each other that is equivalent to a distance between a distal end of the first laser fiber and a distal end of the second laser fiber.

8. The method of claim 7, further comprising:
measuring a dimension of the target based on (i) a known distance between the first collimated beam and the second collimated beam and (ii) the composite image.

9. The method of claim 1, wherein the composite image has a different exposure than either the first image group or the second image group.

10. The method of claim 1, further comprising:
transitioning the medical system to a third configuration, wherein the third configuration comprises (i) the imager being in a second exposure state; and
capturing, via the imager, one or more images of the target comprising a third image group,
wherein the combining further includes the third image group.

11. The method of claim 10, wherein the first configuration comprises (i) the one or more illumination devices being in the first illumination state, and (ii) the first laser fiber and the second laser fiber being in the first laser state, wherein the first image group is captured with the medical system in the first configuration.

12. The method of claim 11, wherein the first laser state includes the first laser fiber and the second laser fiber each being in active states.

13. The method of claim 12, wherein a second configuration of the medical system comprises (i) the one or more illumination devices being in the first illumination state and (ii) the imager being in the first exposure state, wherein the second image group is captured with the medical system in the second configuration.

14. The method of claim 12, wherein the third configuration comprises (i) the one or more illumination devices being in the first illumination state and (ii) the imager being in the second exposure state, and (iii) the first laser fiber and the second laser fiber being in a second laser state different from the first laser state, wherein the third image group is captured with the medical system in the third configuration.

15. The method of claim 14, wherein the first laser fiber is configured to transmit a first collimated beam and the second laser fiber is configured to transmit a second collimated beam, and wherein the first collimated beam emitted from a distal end of the first laser fiber and the second collimated beam emitted from a distal end of the second laser fiber are parallel to each other, maintaining a distance between each other that is equivalent to a distance between a distal end of the first laser fiber and a distal end of the second laser fiber, the method further comprising:
measuring a dimension of the target, based on (i) a known distance between the first collimated beam and the second collimated beam and (ii) the composite image.

16. A method, comprising:
inserting a distal portion of a scope into a bodily orifice or lumen;
positioning the distal portion of the scope adjacent to a first target within the bodily orifice or lumen;
saving a first configuration comprising three parameters, the three parameters including a first exposure parameter, a first illumination parameter, and a first laser parameter;
acquiring a first view of the first target in a field of view of an imager;
determining that the first view with the first configuration does not meet a predetermined quality metric;
adjusting one of the parameters of the first configuration;
saving a second configuration comprising the adjusted parameter of the first configuration and two unadjusted parameters of the first configuration; and
capturing a first image group comprising one or more images of the first target with the second configuration.

17. The method of claim 16, further comprising:
determining a second target from a predefined list of targets;
calculating a third configuration, based on the second target and the second configuration;
acquiring a first view of the second target in the field of view of the imager;
determining that the first view of second target with the third configuration does not meet the predetermined quality metric;
based on the first view of the second target, adjusting one of the parameters of the third configuration;
saving a fourth configuration comprising the adjusted parameter of the third configuration and the two unadjusted parameters of the third configuration, wherein the first view of the second target with the fourth configuration meets the predetermined quality metric; and
capturing a second image group comprising one or more images of the second target with the fourth configuration.

18. The method of claim 17, further comprising:
determining that there are no additional targets from the predefined list of targets;
restoring the saved first configuration; and
generating a first measurement based on the first image group and a second measurement based on the second image group.

19. A medical system comprising:
a scope including a handle and a shaft defining a channel having a distal opening;
a processor;
a light source;
at least one laser source;
a first laser fiber; and
a second laser fiber, wherein each of the first laser fiber and the second laser fiber is coupled to the at least one laser source and extends through the shaft,
wherein a distal end of the shaft includes an imager, the distal opening of the channel, a distal end of the first laser fiber, and a distal end of the second laser fiber, and
wherein the first laser fiber is configured to transmit a first collimated beam onto a target, and the second laser fiber is configured to transmit a second collimated beam on the target,
wherein the processor is configured to adjust (i) a laser parameter for the first laser fiber and the second laser fiber, and (ii) an illumination parameter for the light source, wherein the imager is configured to capture a first image with the at least one laser source in a first laser parameter and the light source in a first illumination parameter, wherein the imager is configured to capture a second image with the at least one laser source in a second laser parameter different from the first laser parameter and the light source in a second illumination parameter different from the first illumination parameter, wherein the processor is configured to combine the first image and the second image into a third image.

20. The medical system of claim 19, wherein the third image has a different exposure than both the first image and the second image.
